(19) United States
(12) Patent Application Publication
(10) Pub. No.: US 2004/0043478 A1
(43) Pub. Date: Mar. 4, 2004

(54) Plastic slides for the fabrication of biochips

(76) Inventors: Li-Wei Hsu, Taichung City (TW); Su-Chen Chang, Taichung City (TW)

Correspondence Address: Kate H. Murashige, Morrison & Foerster LLP, Suite 500, 3811 Valley Center Drive, San Diego, CA, 92130-2332, US

(21) Appl. No.: 10/233,235

(22) Filed: Aug. 29, 2002

(51) Int. Cl. C12M 1/34; B05D 3/00; A61L 2/00

(52) U.S. Cl. 435/287.2; 427/2.11

(57) ABSTRACT

The present invention relates to a plastic slide which after being chemically surface-treated is useful for the immobilization of proteins, peptides or small molecules to its treated surface and thus is useful in the fabrication of biochips. The plastic slide has at least one cavity chamber, wherein the depth of the cavity chambers may be the same or different and generally ranges from less than about 0.1 mm to up to about 0.5 mm. The present invention also relates to a plastic slide made of polystyrene, which after being surface-treated is useful for the immobilization of oligonucleotides or polynucleotides to its treated surface and thus is useful in the fabrication of DNA chips.

### TECHNICAL FIELD OF THE INVENTION

The present invention relates to plastic slides useful in the fabrication of biochips. In particular, the present invention relates to molded plastic slides having a special construction and useful for preparing chemically surface-treated plastic slides, on which microarrays of biological samples can be spotted for the fabrication of biochips.

### BACKGROUND OF THE INVENTION

Glass slide has been conventionally used in the art as a substrate for the fabrication of DNA chips and protein chips. It has been noted, however, that glass slide as a substrate of biochips has several disadvantages. Firstly, glass slide itself is fragile and has to be handled carefully. It is not easy to chemically modify the surface of glass slide in order to successfully immobilize biological, small molecules (such as metabolites from plants or herbs) to said modified surface for binding directly. Glass slide may produce an undesired, high background signal that interferes with the analysis of the biochips that are subsequently treated with a label material. There has been a demand in the art to create a new material or to develop a material that could be easily modified in replace of glass as a biochip substrate.

Recently, efforts in the development of biochips having a plastic substrate have been made, based on the ground that a plastic substrate is not only more economical than a glass slide, but also can be easily molded into a shape as desired by a conventional injection molding process.

WO 00/55627 disclosed a biochip for the detection of target analytes, comprising an array of biologically binding ligands immobilized to a non-fluorescent acrylic support.

WO 00/36145 disclosed a method for making a biochip, comprising grafting biological probes on a conductive polymer.

EP 1 026 259 disclosed a DNA chip comprising a solid carrier and oligonucleotides or polynucleotides fixed on the solid carrier in the presence of a hydrophilic polymer.

Though plastic slides have been used for the fabrication of DNA chips, they are flat without the presence of any cavity chambers or protrusions on the top surface thereof, and also they produce an undesired, high background signal that interferes with the analysis of the biochips. Up to date, it has not been successful in the art to use a plastic slide with its surface having been chemically modified, for immobilizing proteins, peptides or small molecules to its treated surface, preferably in microarrays, especially at a condition that those proteins, peptides or small molecules have not been modified.

In addition, there was no teaching or suggestion in the art that a plastic slide made of polystyrene and having cavity chambers on its top surface could be treated in one step on its surface with a simple reagent, whereby oligonucleotides or polynucleotides could be immobilized, preferably in microarrays, to its treated surface.

### SUMMARY OF THE INVENTION

The present invention relates to a plastic slide that after being chemically surface-treated is useful for the immobilization of proteins, peptides or small molecules to its surface. The plastic slide of the present invention thus is useful in the fabrication of biochips. The plastic slide has at least one cavity chamber, wherein the depth of the cavity chambers may be the same or different and generally ranges from less than about 0.01 mm to up to about 0.5 mm.

The present invention also relates to a plastic slide made of polystyrene, which after being chemically surface-treated is useful for the immobilization of oligonucleotides or polynucleotides to its treated surface and thus useful for the fabrication of DNA chips, wherein the plastic slide has at least one cavity chamber, with the depth of the cavity chambers being the same or different, and the depth of the cavity chambers ranging from less than about 0.01 mm to up to about 0.5 mm.

### BRIEF DESCRIPTION OF THE FIGURES

FIG. 1 shows the perspective of a plastic slide which is a prefelTed embodiment of the present invention.

FIG. 2A shows the fluorescent images of a surface-treated, Cy3-SA/Cy5-SA printed plastic slide of the present invention, in which the green fluorescent spots indicate the Cy3-SA-printed spots, and the red ones the Cy5-SA-printed spots.

FIG. 2B shows the fluorescence images of a conventional plastic slide.

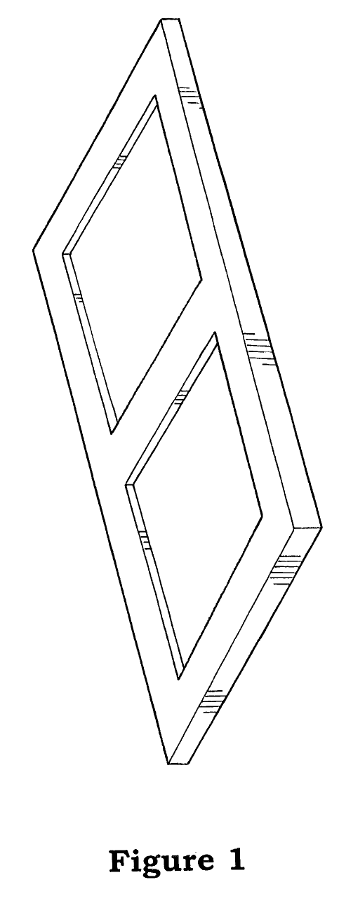

### DETAILED DESCRIPTION OF THE INVENTION

The present invention relates to a plastic slide that after being chemically surface-treated is useful for the direct immobilization of proteins, peptides or small molecules to its treated surface for the fabrication of biochips, wherein said plastic slide has at least one cavity chamber, with the depth of the cavity chambers being the same or different, and the depth of the cavity chambers generally ranging from less than about 0.01 mm to up to about 0.5 mm. Biochips made of the inventive plastic slide can be used as a platform for shotgun screening for biologically active ingredients in a target-directed manner for achieving a high throughput.

The material of the plastic slide used in the present invention may be a homopolymer or copolymer, which is made of one or more monomers selected from the group consisting of ethylene, haloethylene, propylene, halopropylene, acrylate, methacrylate, butadiene, acrylonitrile, norbornene and styrene, wherein a polymer of styrene is preferred. Also included in the material of the plastic slide of the present invention is polycarbonate. In practice, the plastic slide is comparable in size to the ones conventionally used in the art within a microarrayer and a laser scanner.

An advantage of using the inventive plastic slide having a special construction resides in that when compared with a conventional flat plastic slide, a much lower background signal that interferes with the analysis of the biochips is produced.

Another advantage of using a plastic slide in the present invention resides in that there exist a variety of chemicals that can be used for modifying or treating the surface of a plastic slide, whereby not only macromolecules (such as proteins and DNA) but also small molecules (such as metabolites from plants or herbs) can be immobilized to the surface of the plastic slide. Further, the plastic slide of the present invention can be molded in a way to contain preferably at least two cavity chambers, whereby at least two different chemical reactions can be carried out simultaneously in said cavity chambers on the same plastic slide. The depth of the cavity chambers may be the same or different and generally ranges from less than about 0.01 mm to up to about 0.5 mm. There also may be two bars molded respectively at the opposite sides of each chamber for supporting a glass lid which functions to prevent the evaporation or loss of the solution from the chamber.

In the preparation of the chemically surface-treated plastic slide, the raw plastic slide as mentioned above may be pretreated with a polyfunctional aldehyde followed by soaking in a solution of NH₂ group(s)-providing precursor, whereby the pretreated plastic slide contains active amino groups on its surface. The NH₂ group(s)-providing precursor may be organic or inorganic, and may be selected from the group consisting of NH₄OH, primary amine, secondary amine and tertiary amine, wherein the aliphatic or aromatic part of the primary amine, secondary amine and tertiary amine may be useful as an additional spacer arm. Among the NH₂ group(s)-providing precursors, NH₄OH and the primary amine that directly provide a free NH₂ group are preferred.

The pretreated plastic slide is further coated with a layer of a polyfunctional molecule (e.g. a polyfunctional epoxide) that is useful as a spacer for producing the chemically surface-treated plastic slide. In function, the polyfunctional epoxide will act for linking the desired components in test samples spotted on the chemically surface-treated plastic slide, preferably in microarrays. The active epoxy groups on one end of the polyfunctional epoxide react with the amino groups on the surface of the pretreated plastic slide, while the active epoxy groups on the other end of the polyfunctional epoxide react with or absorb the desired components in the test samples. In particular, those components in the test samples that contain free hydroxyl, sulfhydryl or amino groups can form a covalent bond with the active epoxy groups on the other end of the polyfunctional epoxides, and consequently are attached to the chemically surface-treated plastic slide. The polyfunctional epoxides preferably contain a long chemical chain of 6 to 24 carbon atoms, whereby the desired components would not directly bind to the pretreated plastic slide. The binding of the desired components to the chemically surface-treated plastic slide is persistent, even after stringent stripping. In the present invention, not only macromolecules (such as proteins and polypeptides) but also small molecules (such as metabolites from plants or herbs), regardless of being homogeneous or heterogeneous, can be immobilized to the surface of the chemically surface-treated plastic slide.

Based on the above, the preparation of the chemically surface-treated plastic slide comprises the steps of preparing a raw plastic slide provided with cavity chambers, pretreating the raw plastic slide with a polyfunctional aldehyde followed by soaking in a solution of NH₂ group(s)-providing precursor (preferably, aqueous ammonia), and coating the surface of the pretreated plastic slide with a polyfunctional molecule (preferably, a polyfunctional epoxide).

The chemically surface-treated plastic slide is useful in the preparation of biochips, wherein microarrays of spots containing homogeneous or heterogeneous samples can be immobilized to the surface of the chemically surface-treated plastic slide. The biochips thus obtained can be used for screening for the desired components contained in the spotted test samples based on a target-directed strategy. The screening method may comprise the steps of loading the biochips with a labeled probe(s)-containing solution for conducting a chemical reaction (wherein each of the chambers may be covered by a glass lid for preventing the evaporation of the labeled probe(s)-containing solution), and imaging and identifying the spots that react with or bind to the labeled probe by using an apparatus, e.g. a laser scanner. The label within the probes may be a dye or a radioactive material. Further, the probes used for the hybridization may be homogeneous or heterogeneous, known targets based on a defined molecular mechanism, which may be, for example, small molecules, competitive ligands, or antibodies against, for example, the desired receptors, enzymes, or proteins.

The present invention also relates to a plastic slide made of polystyrene, which after being chemically surface-treated is useful for the immobilization of oligonucleotides or polynucleotides to its treated surface, preferably in microarrays, and thus is useful in the fabrication of DNA chips. Said plastic slide has at least one cavity chamber, wherein the depth of the cavity chambers may be the same or different and generally ranges from about 0.01 mm to up to about 0.5 mm. The DNA chips made of the surface-treated polystyrene slide of the present invention are useful as a platform for the detection of the presence of desired DNAs in the sample spots on its treated surface under hybridization conditions with a labeled probe.

The polystyrene slide in the present invention also may have a size comparable to the ones conventionally used in the art within a microarrayer and a laser scanner. The polystyrene slide also may be molded in a way to contain preferably at least two cavity chambers as mentioned above, whereby at least two different hybridization reactions can be carried out simultaneously in said cavity chambers on the same polystyrene slide. In practice, the depth of the cavity chambers may be the same or different, and may range from less than about 0.01 mm to up to about 0.5 mm. There also may be two bars molded respectively at the opposite sides of each chamber for supporting a glass lid which functions to prevent the evaporation or loss of the solution from the chamber.

In the modification of the surface of a polystyrene slide, a raw polystyrene slide is coated with a reagent comprising a NH₄⁺ group-free buffer containing positive charges-providing polymers (e.g. polylysine) under a stronger alkaline condition. The NH₄⁺ group-free buffer may be a carbonate, phosphate or citrate buffer. The stronger alkaline condition may be at a pH in the range of pH 9 to 11. The advantage in the preparation of the surface-treated polystyrene slide of the present invention resides in that a simple reagent in just one coating step is needed.

The DNA chips thus obtained can be used for the detection of the presence of desired DNAs in the spotted test samples under hybridization conditions with a labeled probe. Such detection method may comprise the steps of loading the DNA chips with a labeled probe(s)-containing solution for conducting a hybridization reaction (wherein each of the chambers may be covered by a glass lid for preventing the evaporation of the labeled probe(s)-containing solution), and imaging and identifying the spots that bind to the labeled probe(s) with an apparatus, e.g. a laser scanner. Also as mentioned above, the label within the probe(s) may be a dye or a radioactive material.

In effect, both of the biochips and DNA chips made of the surface-treated plastic slide of the present invention show a much weaker fluorescence background than those biochips made of a glass slide.

The following Examples are intended to illustrative the invention without limiting the scope thereof.

### EXAMPLE

Labeling of Strapavidin with Fluorophore

Cy3-strapavidin (Cy3-SA) and Cy5-strapavidin (Cy5-SA) were prepared using FluoriLink™Cy3™ bifunctional reactive dye and FluoriLink™Cy5™ bifunctional reactive dye (Amersham) according to the recommended protocols. The uncoupled fluorescence residue was then removed by gel filtration chromatography using a Bio-Gel P2 (Bio-Rad) column (0.5 cm×10 cm) equilibrated in TBST (50 mM Tris.HCl, pH 7.35 containing 0.15 M NaCl). The final concentration was estimated to be 0.5 mg/ml.

Pretreatment of Plastic Slides and Preparation of Coated Plastic Slides

Molded plastic slides and flat plastic slides were made of a polymer of styrene. The molded plastic slides comprised two cavity chambers and were comparable in size with the regular glass slides used for microscope or laser scanner, wherein the depth of each of the cavity chambers is 0.05 mm. The flat plastic slides were also comparable in size with the regular glass slides used for microscope or laser scanner.

Both the molded and flat slides were first immersed in an aqueous 0.4% glutaldehyde solution (pH 5.0) for 4 hours at room temperature, followed by washing with water and then soaking in 3M NH₄OH (pH 11.0) at 60° C. for 4 hours. The resultant plastic slides were then treated with 100 mM of 1,4-butanediol diglycidyl ether (pH 11.0) at 37° C. overnight. The plastic slides obtained were washed with 0.1 M aqueous NaHCO₃ (pH 8.0) once and double-distilled water four times, optionally followed by being stored in double-distilled water at 4° C. for 4˜16 hours. The resultant slides were dried in a safety cabinet at room temperature prior to use.

Robotic Arraying of Cy3-SA and Cy5-SA

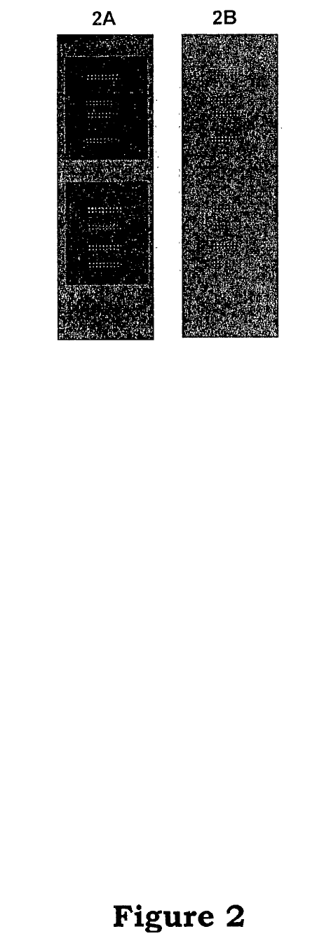

The robot, MicroGrid II (BioRobitics), with 0.4 mm solid pin was used for printing the diluted Cy3-SA and Cy5-SA to defined locations on the molded and flat plastic slides. Both the Cy3-SA and Cy5-SA proteins were two-fold serially diluted into 30% (10-60%) DMSO/0.1M carbonate buffer, pH 9.5 (pH7.5˜11), from the concentration of 20 μg/ml to 39 ng/ml. The pin used to deliver the compounds was washed with double distilled water for 2 seconds and then in 70% Ethanol for 2 seconds, followed by drying under a stream of hot air for 2 seconds before printing each sample. Following printing, both the molded and flat plastic slides were incubated at room temperature for 2 hours and then immersed in 1M ethanolamine. The slides were subsequently washed 3 times with an aqueous solution of TBST 50 mM Tris.HCl, 0.15M NaCl, 0.05% Tween 20, followed by rinsing with doubled distilled water three times and drying at 37° C. for 5 minutes and then scanned using GenePix 4000A slide scanner (Axon Instruments). The images were then analyzed by GenePix 3.0 Software. The results are shown in FIGS. 2A and 2B, wherein the printed samples in FIGS. 2A and 2B are serially diluted two-fold from columns 1 to 10 (left to right).

The concentrations in duplicates of each of samples in row are indicated as following:

Row (a) & (b):

Cy5-SA: 20, 10, 5, 2.5, 1.25, 0.625, 0.312, 0.156, 0.078, 0.039 μg/ml

Row (c) & (d):

Cy3-SA: 20, 10, 5, 2.5, 1.25, 0.625, 0.312, 0.156, 0.078, 0.039 μg/ml

What is claimed is:

1. A plastic slide which after being chemically surface-treated is useful for the immobilization of proteins, peptides or small molecules to its treated surface, wherein the plastic slide has at least one cavity chamber, with the depth of the cavity chambers being the same or different and generally ranging from less than about 0.01 mm to up to about 0.5 mm.

2. The plastic slide as claimed in claim 1, wherein the plastic slide is formed of a material, which is a polycarbonate, or a homopolymer or copolymer that is made of one or more monomers selected from the group consisting of ethylene, haloethylene, propylene, halopropylene, acrylate, methacrylate, butadiene, acrylonitrile, norbornene and styrene.

3. The plastic slide as claimed in claim 1, wherein the plastic slide is formed of a polymer of styrene.

4. The plastic slide as claimed in claim 1, wherein the plastic slide has at least two cavity chambers.

5. The plastic slide as claimed in claim 4, wherein the depth of the cavity chambers may be the same or different, and ranges from about 0.01 mm to about 0.5 mm.

6. The plastic slide as claimed in any one of claims 1 to 5, which is useful in the fabrication of biochips.

7. The plastic slide as claimed in any one of claims 1 to 5, which may be surface-treated by pretreating with a polyfunctional aldehyde followed by soaking in a solution of NH₂ group(s)-providing precursor and subsequently coating the plastic slide.

8. The plastic slide as claimed in claim 7, wherein the polyfunctional aldehyde is glutaldehyde.

9. The plastic slide as claimed in claim 7, wherein the NH₂ group(s)-providing precursor is NH₄OH.

10. The plastic slide as claimed in claim 7, wherein the coating is formed of polyfunctional molecules.

11. The plastic slide as claimed in claim 7, wherein the polyfunctional molecule is a polyfunctional epoxide containing at least one epoxy group at each of its ends.

12. The plastic slide as claimed in claim 11, wherein the epoxy group(s) at one end of the polyfunctional epoxide react with the amino group(s) on the surface of the pretreated plastic slide.

13. The plastic slide as claimed in claim 11, wherein the epoxy group(s) at the other end of the polyfunctional epoxide react with the free hydroxyl, sulfhydryl or amino groups of the proteins, peptides or small molecules.

14. The plastic slide as claimed in claim 11, wherein the polyfunctional epoxide contains a long chemical chain of 6 to 24 carbon atoms.

15. The plastic slide as claimed in claim 1, wherein the proteins, peptides or small molecules are homogeneous or heterogeneous.

16. A polystyrene slide which after being surface-treated is useful for immobilizing thereto oligonucleotides or polynucleotides, wherein the plastic slide has at least one cavity chamber, with the depth of the cavity chambers being the same or different and ranging from less than about 0.1 mm to up to about 0.5 mm.

17. The polystyrene slide as claimed in claim 16, which can be surface-treated by applying to the surface thereof a reagent comprising a NH₄⁺ group-free buffer containing positive charges-providing polymers under an alkaline condition.

18. The polystyrene slide as claimed in claim 17, wherein the positive charges-providing polymer is polylysine.

19. The polystyrene slide as claimed in claim 17, wherein the NH₄⁺ group-free buffer is selected from the group consisting of a carbonate, phosphate and citrate buffer.

20. The polystyrene slide as claimed in claim 17, wherein the alkaline condition is in the range of pH 9 to 11.

21. The polystyrene slide as claimed in claim 16, wherein the polystyrene slide has at least one cavity chamber.

22. The polystyrene slide as claimed in claim 21, wherein the depth of the cavity chambers may be the same or different, and ranges from about 0.01 mm to about 0.5 mm.
